(19) United States
(12) Patent Application Publication
(10) Pub. No.: US 2009/0188510 A1
(43) Pub. Date: Jul. 30, 2009

(54) Treatment of obstructive sleep disorders

(76) Inventor: Harold D. Palmer, Monument, CO (US)

Correspondence Address: LINDA FLEWELLEN GOULD, 1665 BRIARGATE BLVD. #101, COLORADO SPRINGS, CO, 80920, US

(21) Appl. No.: 12/011,078

(22) Filed: Jan. 24, 2008

(51) Int. Cl. A61F 5/56

(52) U.S. Cl. 128/848

(57) ABSTRACT

The present invention provides a method and an apparatus for treating sleep disorders related to partial or complete obstruction of air passageways by the tongue during sleep, such as snoring and sleep apnea. A mouthpiece is inserted in a user's mouth, so that a radial portion of the mouthpiece is held between the user's teeth and lips. An extension portion of the mouthpiece extends from the radial portion forward, through the user's lips, ending in a tip in which is formed a suction hole. A vacuum bladder snugly fits on the tip of the extension portion of the mouthpiece, so that pinching the vacuum bladder expels air from the bladder through the suction hole and into the extension portion of the mouthpiece. When the mouthpiece is positioned in a person's mouth, releasing the compressed bladder creates a vacuum suitable for comfortably holding the person's tongue within a tongue cavity inside the extension portion of the mouthpiece.

### BACKGROUND OF THE INVENTION

1. Field of the Invention

The present invention relates to a method of and apparatus for treating sleep disorders associated with partial or complete obstruction of air passageways by the tongue, such as snoring and sleep apnea.

2. Description of the Prior Art

Clearly, the average person is most productive and content when he or she has slept well. In fact, insufficient sleep and resulting fatigue has been shown to cause inefficiency in the workplace and, in some situations, dangerous conditions.

A number of sleep disorders can be linked to obstruction of air passageways by the tongue. When a person is awake, the tongue naturally allows air to pass into and out of the lungs. However, the tongue is relaxed during sleep, and this may result in partial or complete obstruction of air passageways in some people. As air is drawn into the lungs during respiration, the tongue may be pulled against the posterior wall of the pharynx. This may result in snoring sounds, possibly disturbing the sleep of the person who is snoring or other family members. More dangerously, the tongue, when positioned against the posterior wall of the pharynx, may impede air from entering the lungs. This condition is known as sleep apnea. Sleep apnea may cause increases in blood carbon dioxide level, increases in blood pressure and pulse rate, and possibly arrhythmias. Ultimately, the body reacts to lack of oxygen by waking up, allowing the tongue muscle to resume its non-relaxed state and thus unsealing the air passageways. But the person who suffers untreated sleep apnea finds it impossible to get sufficient sleep in a typical night's rest because of repeated waking episodes.

A number of devices have been used to treat obstructive sleep disorders. Many of these devices pull the tongue forward during sleep, to avoid obstruction of airways. While maintaining the tongue in a forward position is an effective way to prevent sleep apnea, finding a way to keep the tongue in this position in a manner that is comfortable enough to permit sleep has been a challenge.

The anti-snoring device of U.S. Pat. Nos. 4,169,473 and 4,304,227 to Samelson consists of a device which is held in the mouth to completely block oral flow of air. Clearly, this device could not be used by someone who had a permanent obstruction of nasal passages, or even a bad cold that made breathing through the nose difficult. Furthermore, the tongue may not be held in place for a sufficient period of time to permit an uninterrupted night's sleep by the Samelson device, since the only suction created is from the tongue forcing its way into a cavity of the device. Similarly, the device of U.S. Pat. No. 4,676,240 to Gardy does not provide any manner of holding the tongue in a chamber other than the negative pressure created by pushing the tongue into that chamber.

A device taught in U.S. Pat. No. 5,154,184 to Alvarez holds the tongue in a forward position by inserting the tongue in a receptacle with a narrowed region that tightly fits on the tongue, possibly causing some discomfort. A similar mechanism is described in U.S. Pat. No. 5,465,734 and U.S. Pat. No. 5,649,540 to Alvarez et al., which allows additional suction to hold the tongue in a forward position by connecting a tube from an external source of positive pressure to the device.

The mouthpiece described in U.S. Pat. No. 6,494,209 to Kulick also relies on an external source of pressure to maintain the tongue in a forward position within the mouthpiece. Although this design is useful, the device is cumbersome because of the external air hose and pressure source. The device cannot be used when sleeping away from a source of electricity to power the pressure source and is difficult to transport when the user is traveling. The external tube may also be pulled by any motion of the user while sleeping, possibly disengaging the source of pressure or simply getting in the way of turning over in bed. Finally, a source of external pressure provided through a tube leads to saliva being pulled into the tube, which can clog the tubing through which suction is provided and is difficult to clean.

In a related field, U.S. Pat. No. 3,295,519 to Gerber teaches a mouth exerciser for use in juveniles to prevent thrusting of the tongue forward during swallowing. Therefore, the Gerber device discourages the forward movement of the tongue, contrary to the claimed invention.

While each of these devices is useful for its intended purpose, none effectively enables a user to position the user's tongue in a forward position during a full night's sleep to avoid the tongue obstructing air passageways with a comfortable mouthpiece that requires no outside source of vacuum.

### SUMMARY AND OBJECTS OF THE INVENTION

A primary object of the present invention is to provide a method and apparatus for relieving the symptoms of snoring and sleep apnea that are associated with a user's tongue partially or completely blocking air passageways during sleep.

Another object of the present invention is to provide such an apparatus which can be easily transported and used, in a location that may not have electricity available, since the device does not rely on an external source of vacuum

Yet another object of the present invention is to provide such an apparatus which efficiently and comfortably holds the user's tongue in a forward position throughout a night's sleep.

These objects are achieved by a device consisting of a mouthpiece with a radial portion suitable for fitting between a user's lips and mouth, an extension portion suitable for extending forward between the user's lips inside which the user's tongue can be held by suction, and a vacuum bladder permanently attached to the tip of the extension portion to provide suction.

The radial portion of the mouthpiece has a generally semicircular shape which will comfortably fit between the user's lips and teeth, similar to a mouthpiece used with a snorkel. An aperture is formed in a central, forward-most area of the radial portion of the mouthpiece. This aperture is of sufficient size to allow a user's tongue to extend forward between the user's upper and lower teeth, through the aperture, and between the user's lips. The extension portion of the mouthpiece surrounds that aperture, and extends forward from the radial portion. Thus, the extension portion fits between the user's lips when the radial portion is held in the user's mouth. The hollow inside of the extension portion forms a tongue cavity in which the user's tongue can be comfortably held during sleep. When the tongue is held within the extension portion, the tongue cannot physically block airways at the back of the user's mouth, and cannot cause the snoring noises which result from a relaxed tongue fluttering against the posterior wall of the pharynx.

The tongue is held in a forward position within the extension portion by a vacuum created by the vacuum bladder which is permanently attached to the tip of the extension portion. When air is expelled from the vacuum bladder by pinching it, that air is pushed from the vacuum bladder through a suction hole in the tip of the extension portion which is surrounded by the vacuum bladder. While the bladder is held in a pinched position, the user places his or her tongue within the cavity of the extension portion so that the forward most point of the tongue gently touches the suction hole at the tip of the extension portion. When the pinch bladder is released, the tongue prevents air from moving back into the bladder through the suction hole. As a result, the vacuum bladder remains partially compressed, and a positive vacuum holds the tongue within the extension portion.

Unlike devices known in the prior art, the pinch bladder provides an ideal amount of suction, measured at approximately 20-115 mmHg. This suction is sufficient to hold the tongue in place but not powerful enough to cause the tongue to become uncomfortable or numb. If the vacuum is released unintentionally, a simple pinch and release of the vacuum bladder restores the grip on the tongue almost immediately.

Releasing the tongue and removing the mouthpiece from the mouth is simply achieved by pinching the vacuum bladder to release the vacuum which was created by the bladder attempting to pull air from the extension portion.

At least one breathing opening is provided in the mouthpiece to permit oral breathing while the device is in place within the mouth. One or more breathing openings, such as slits or holes, are most beneficially formed in the extension portion to permit air to flow through the extension portion and into the mouth during normal respiration. By placing the breathing opening in the extension portion, the user can breathe through his mouth or nose.

In this manner, the device provides more efficient suction to hold the tongue forward than is possible with devices that do not provide a source of suction other than the tongue pushing air out of a tongue cavity. This suction is provided without a cumbersome and potentially noisy external source of vacuum. Furthermore, suction is directed to multiple surface areas of the tongue, holding the tongue in a much more comfortable manner than is possible using a single point of suction or using mechanical holding devices. These objectives are provided in a self-contained, easy-to-transport, and easy-to-clean device.

### BRIEF DESCRIPTION OF THE DRAWINGS

FIG. 1 is a perspective side view of a device for treating obstructive sleep disorders, according to the present invention.

FIG. 2 is a perspective view of a device for treating obstructive sleep disorders, viewed from the back end, according to the present invention.

FIG. 3 is a top, planar view of a device for treating obstructive sleep disorders, according to the present invention, showing the position of a user's tongue held by the device.

FIG. 4 is a sagittal view of a user's mouth and oral cavity when using the device of the present invention.

FIG. 5 is a side view of the device of the present invention shown inserted in a user's mouth prior to creating a vacuum.

FIG. 6 is a side view of the device of the present invention shown inserted in a user's mouth as a vacuum bladder is compressed.

FIG. 7 is a side view of the device of the present invention shown inserted in a user's mouth, holding the user's tongue in a forward position.

In the drawings, the following legend has been used:

| | |
|---|---|
| 10 | Device for treating sleep disorders |
| 12 | Mouthpiece |
| 14 | Radial portion of mouthpiece |
| 15 | Front surface of radial portion of mouthpiece |
| 16 | Extension portion of mouthpiece |
| 17 | Back surface of radial portion of mouthpiece |
| 18 | Upper lip ledge |
| 20 | Breathing opening |
| 22 | Upper teeth ledge |
| 24 | Tongue cavity |
| 25 | Aperture in radial portion |
| 26 | Top face of extension portion |
| 28 | Bottom face of extension portion |
| 32 | Suction hole in tip of extension portion |
| 34 | Vacuum bladder |
| 36 | Upper area of radial portion |
| 38 | Lower area of radial portion |
| 40 | Tip of extension portion |
| 42 | Opening of vacuum bladder |
| 50 | Upper teeth |
| 52 | Lower teeth |
| 54 | Tongue |
| 56 | Upper lip |
| 58 | Lower lip |
| 60 | Oral cavity |
| 62 | Uvula of soft palate |
| 64 | Posterior wall of pharynx |
| 65 | Breathing path |

### DESCRIPTION OF THE PREFERRED EMBODIMENT

The present invention concerns a method and apparatus for treating obstructive sleep disorders by effectively and comfortably holding a user's tongue in a forward position during sleep. In the following description, numerous specific details are set forth in order to provide a thorough understanding of the present invention. It will be obvious, however, to one skilled in the art that the present invention may be practiced without these specific details. Some well-known methods and structures have not been set forth in order not to unnecessarily obscure the description of the present invention.

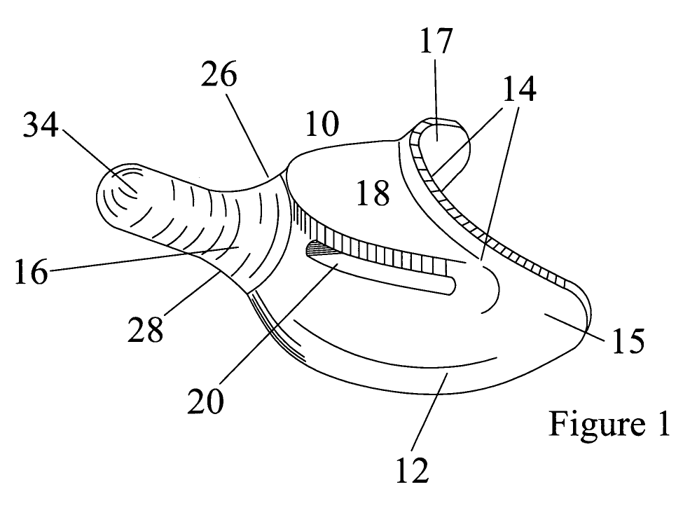

The device for treating obstructive sleep disorders 10 claimed herein includes mouthpiece 12 with a radial portion 14 and an extension portion 16, as shown in FIG. 1. The mouthpiece 12 can advantageously be made of any material which can be comfortably and safely held in the mouth, such as flexible, non-toxic, moldable plastic.

As best shown in FIG. 1, the radial portion 14 of the mouthpiece 12 has a radial or convex shape suitable for fitting between a user's lips 56, 58 and teeth 50, 52. The front surface 15 of the radial portion 14 is designed to comfortably engage the user's lips 56, 58, while the back surface 17 of the radial portion 14 fits snugly against the user's teeth 50, 52.

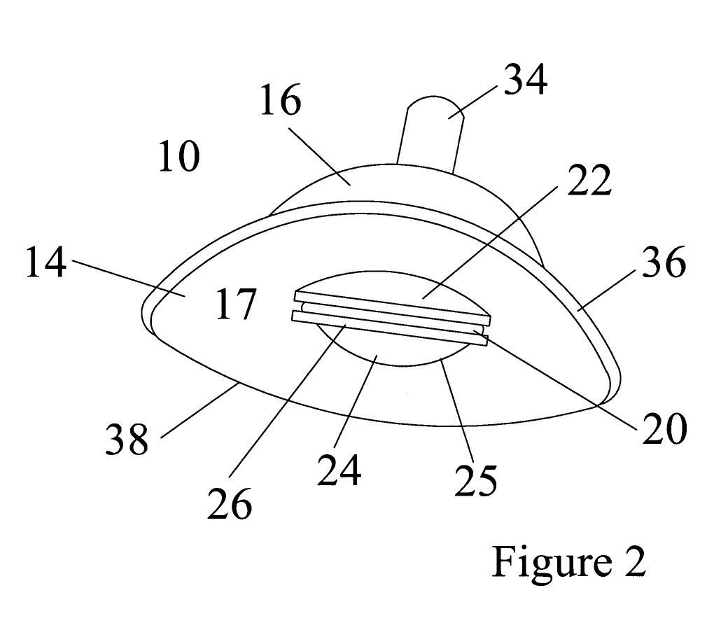

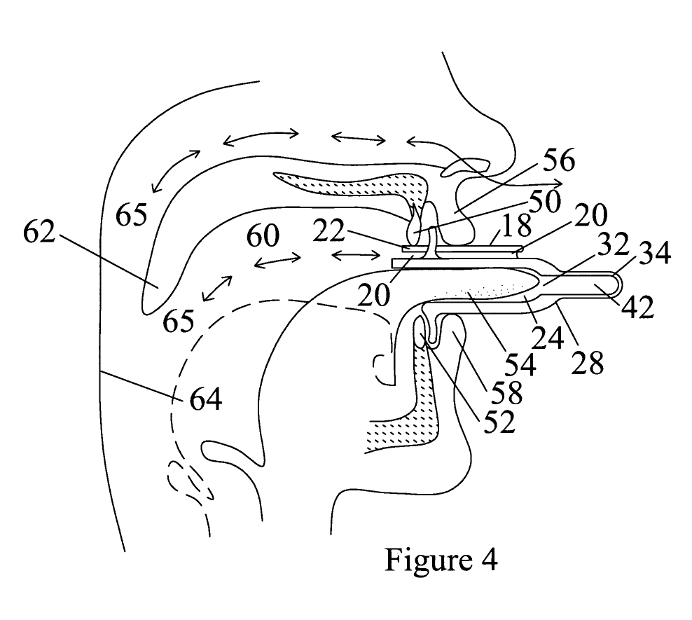

As best shown in FIG. 2, an aperture 25 is formed in a central, forward most area of the radial portion 14. This aperture 25 is sized and shaped to comfortably receive a user's tongue 54, allowing the tongue 54 to be inserted between the upper teeth 50 and lower teeth 52, as shown in FIG. 4. The hollow extension portion 16 surrounds the aperture 25, extending forward from the radial portion 14. Thus, the interior of the extension portion 16 forms a tongue cavity 24 in which the user's tongue 54 can be comfortably held during sleep.

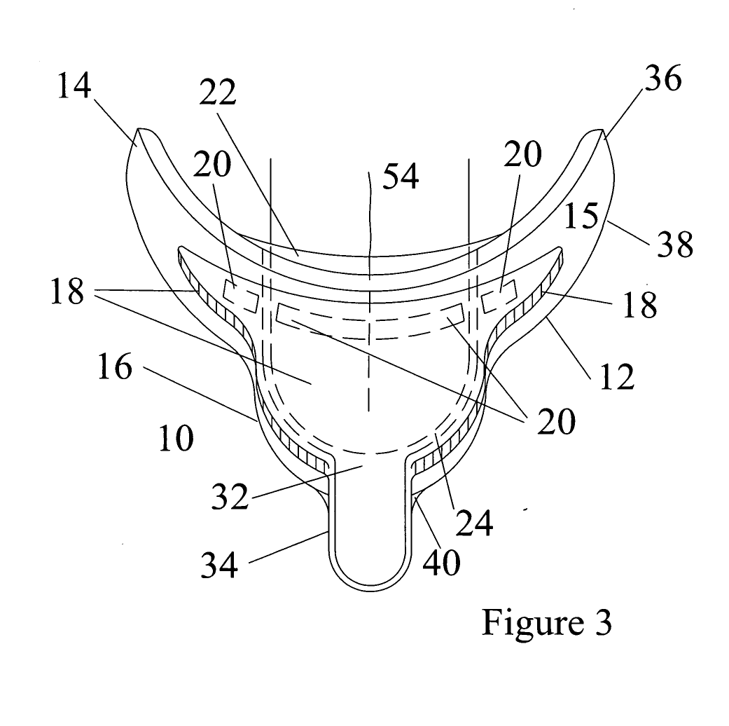

The extension portion 16 extends to a tip 40. The extension portion 16 may be tapered toward the tip 40, or may extend forward to the tip 40 without tapering. A suction hole 32 is formed in the tip 40, as shown in FIG. 3. A vacuum bladder 34 is attached to the tip 40, preferably in a permanent manner so that the vacuum bladder 34 conveniently forms a unified mechanism with the radial portion 14 and extension portion 16 of the mouthpiece 12. The opening 42 of the vacuum bladder 34 surrounds the tip 40 so that air entering or being expelled from the vacuum bladder 34 must pass through the suction hole 32 in the tip 40.

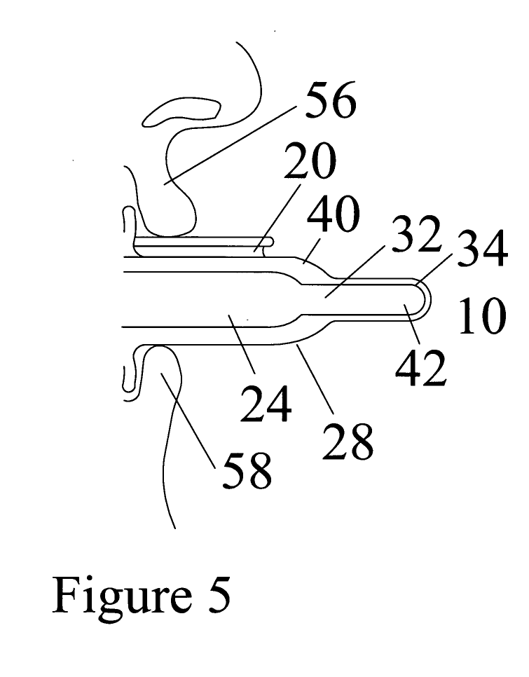

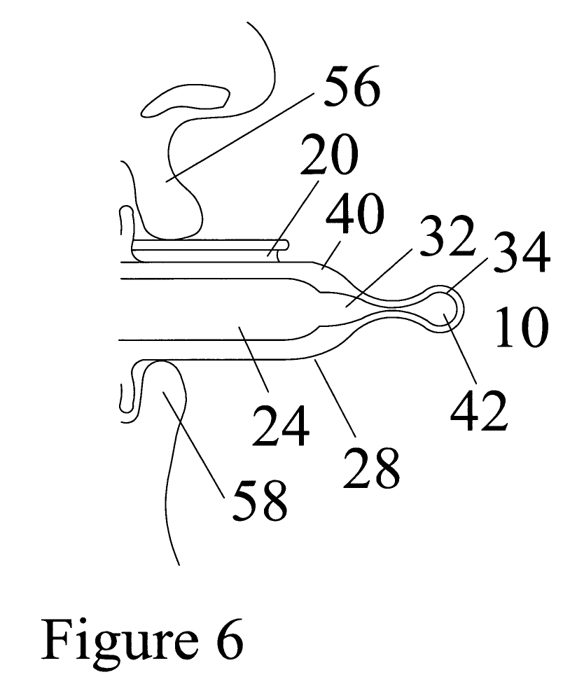

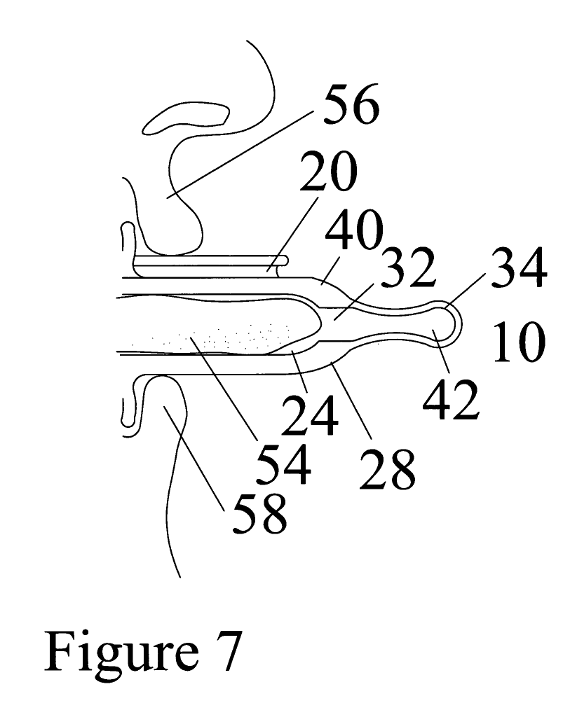

The mouthpiece 12 is inserted into a user's mouth between the user's upper lip 56 and lower lip 58 as shown in FIG. 5. To most efficiently create a vacuum which will hold the tongue 54 forward, the vacuum bladder 34 is compressed, as shown in FIG. 6, to expel air (not shown) from that bladder 34 through the suction hole 32. The vacuum bladder 34 is easily compressed between the user's thumb and finger (not shown). While the bladder 34 is compressed, the user's tongue 54 may be inserted into the tongue cavity 24 of the extension portion 16 through the aperture 25 between the upper area 36 of the radial portion and the lower area 38 of the radial portion, as shown in FIG. 2. While the tongue 54 is inserted into the extension portion 16, as shown in FIG. 7, the vacuum bladder 34 is released, creating suction in the direction of the vacuum bladder 34, holding the tongue 54 within the tongue cavity 24 of the extension portion 16. The vacuum bladder 34 creates suction around the front portion of the tongue 54 held within the tongue cavity 24, comfortably applying pressure to all of the tongue 54 within that cavity 24. As a result, the tongue 54 is pulled upward and forward within the oral cavity 60 from its typical rest position shown in broken lines in FIG. 4, to the forward position shown in FIGS. 3 and 4. In this manner, the tongue 54 is held away from the posterior wall of the pharynx 64, and does not contact the uvula of the soft palate 62, so that airways are not blocked and no snoring noises are created.

One or more breathing openings 20 may beneficially permit the user to breathe through the oral cavity 60 even when the mouthpiece 12 is in place, allowing oral breathing as well as breathing through the nose, along the breathing paths 65 shown in FIG. 4. Each breathing opening 20 may consist of a slit or hole formed in the radial portion 14 or the extension portion 16. It is beneficial to create at least one breathing opening 20 in the top face 26 of the extension portion 16, as best shown in FIG. 4.

A lips ledge 18, as shown in FIG. 1, supports the user's upper lip 56, and prevents the upper lip 56 from blocking a breathing opening 20 located below the lips ledge 18. A teeth ledge 22, as shown in FIG. 4, extends backwards from the back surface 17 of the upper area 36 of the radial portion 14 of the mouthpiece 12. This teeth ledge 22 beneficially supports the user's upper teeth 50 and separates the upper teeth 50 from the breathing opening 20 and the lower teeth 52, to facilitate placement of the tongue 54 under the breathing opening 20, between the upper teeth 50 and lower teeth 52.

Thus, the present invention provides a method of effectively and comfortably holding a user's tongue in a forward position during sleep, to treat obstructive sleep disorders such as snoring and sleep apnea. Because the claimed device is easily transportable, easily cleanable with soap and water, and does not rely on an external source of vacuum, the device can be used in many different settings, including camping in a tent, temporary lodging for a traveler, or in the user's usual bed. Unlike devices known in the prior art, the tongue is held by built-in suction applied to the user's tongue, precluding the discomfort associated with a device which applies suction from an external vacuum source. The built-in suction provided by the pinch bladder 34 is significantly more effective than suction created only by placing the tongue in a cavity of devices known in the prior art. Furthermore, the device does not rely on bite blocks to maintain its position in the mouth, which bite blocks may be particularly uncomfortable for a person with missing teeth. Because the device is so comfortable and an effective long-lasting, internal source of vacuum is provided, the user is more likely to get a good night's sleep than would be possible with devices known in the prior art.

Although the present invention has been described in terms of the presently preferred embodiment, it is to be understood that such disclosure is purely illustrative and is not to be interpreted as limiting. Consequently, without departing from the spirit and scope of the invention, various alterations, modifications, or alternative applications of the invention will, no doubt, be suggested to those skilled in the art after having read the preceding disclosure. Accordingly, it is intended that the following claims be interpreted as encompassing all alterations, modifications, or alternative applications as fall within the true spirit and scope of the invention.

I claim:

1. A device for holding a user's tongue in a forward position to treat sleep disorders, comprising:
a. a mouthpiece having a radial portion sized and shaped to fit between a user's teeth and lips, having a front surface suitable for positioning adjacent to a user's lips, a back surface suitable for positioning adjacent to a user's teeth, and having a hollow extension portion extending forward from said radial portion surrounding an aperture in said radial portion suitable for receiving a forward portion of a user's tongue,
b. said mouthpiece having at least one breathing opening formed therein,
c. said extension portion having a forward most tip in which is formed a suction hole, and
d. compressible vacuum bladder snugly surrounding said tip of said extension portion, so that compressing said vacuum bladder expels air from said bladder through said suction hole of said extension portion and creates a vacuum which holds a forward portion of the user's tongue in said hollow extension portion.

2. A device according to claim 1, further comprising teeth ledge extending from said back surface of said mouthpiece suitable for placing between a user's upper teeth and lower teeth.

3. A device according to claim 2, in which said teeth ledge extends backwards from said back surface above said aperture.

4. A device according to claim 2, in which said teeth ledge extends backwards from said back surface below said aperture.

5. A device according to claim 3, in which said teeth ledge extends beneath and adjacent to a user's front teeth when the device is held in user's mouth.

6. A device according to claim 1, further comprising lips ledge extending forward from said radial portion of said mouthpiece above said aperture to support upper lip of user.

7. A device according to claim 1, in which said breathing opening is formed in said hollow extension portion.

8. A device according to claim 1, in which said breathing opening is formed in said radial portion.

9. A device according to claim 6, in which said breathing opening is formed in said radial portion below said lips ledge and above said extension portion.

10. A device according to claim 1, in which said breathing opening is formed in a top face of said extension portion.

11. A device according to claim 1, in which a plurality of breathing openings are formed in said radial portion.

12. A device according to claim 1, in which a plurality of breathing openings are formed in said extension portion.

13. A device according to claim 1, in which at least one breathing opening is formed in said extension portion and at least one breathing opening is formed in said radial portion.

14. A device according to claim 1, in which said extension portion tapers from said radial portion to said forward most tip.

15. A method for holding a user's tongue in a forward position to treat sleep disorders, comprising the steps of:
a. inserting a mouthpiece into the user's mouth, said mouthpiece having a radial portion positioned between the user's teeth and lips, a front surface adjacent to the user's lips, a back surface adjacent to the user's teeth, a hollow extension portion extending forward from said radial portion surrounding an aperture in said radial portion suitable for receiving a forward portion of a user's tongue, and a forward most tip of said extension portion having a suction hole formed therein,
b. compressing a compressible vacuum bladder snugly surrounding said tip of said extension portion, so that said vacuum bladder expels air from said bladder through said tip of said extension portion,
c. inserting a front portion of the user's tongue through said aperture and into said hollow extension portion, and
d. releasing said vacuum bladder to create a vacuum which holds a forward portion of the user's tongue in said hollow extension portion.
